(19) United States
(12) Patent Application Publication
(10) Pub. No.: US 2013/0231413 A1
(43) Pub. Date: Sep. 5, 2013

(54) POLYETHERESTER POLYOLS AND THE USE THEREOF FOR PRODUCING RIGID POLYURETHANE FOAMS

(71) Applicants: Andreas KUNST, Ludwigshafen (DE); Berend ELING, Lemfoerde (DE); Markus SCHUETTE, Osnabrueck (DE); Sebastian KOCH, Lemfoerde (DE); Christian KOENIG, Mannheim (DE); Marc FRICKE, Osnabrueck (DE)

(72) Inventors: Andreas KUNST, Ludwigshafen (DE); Berend ELING, Lemfoerde (DE); Markus SCHUETTE, Osnabrueck (DE); Sebastian KOCH, Lemfoerde (DE); Christian KOENIG, Mannheim (DE); Marc FRICKE, Osnabrueck (DE)

(21) Appl. No.: 13/760,460

(22) Filed: Feb. 6, 2013

Related U.S. Application Data
(60) Provisional application No. 61/605,218, filed on Mar. 1, 2012.

(51) Int. Cl. C07H 13/06; C07H 17/02; C08G 18/08

(52) U.S. Cl. CPC C07H 13/06; C08G 18/14; C07H 17/02
USPC 521/172; 536/116; 536/55; 252/183.11

(57) ABSTRACT

The invention relates to a polyetherester polyol comprising the reaction product of
a1) 5 to 63 wt % of one or more polyols or polyamines or mixtures thereof having an average functionality of 2.5 to 8,
a2) 2 to 50 wt % of one or more fatty acids, fatty acid monoesters or mixtures thereof,
a3) 35 to 70 wt % of one or more alkylene oxides of 2 to 4 carbon atoms.

The present invention relates to polyetherester polyols, polyol mixtures comprising them, to a process for producing rigid polyurethane foams using the polyetherester polyols and the rigid polyurethane foams themselves.

Rigid polyurethane foams are long known and have been extensively described. Rigid polyurethane foams are predominantly used for thermal insulation, for example in refrigeration appliances, means of transport or buildings and also for producing structural elements, especially sandwich elements.

It is important that the rigid polyurethane foams fill the cavities uniformly and without voids in order that bonding to the outer layers is as good as possible to produce a stable structure that ensures good thermal insulation. To prevent foam defects, the time within which the foamable PU reaction mixture is introduced into the cavity to be insulated has to be short. It is typically low-pressure or preferably high-pressure machines that are usually used to foam out such articles.

A comprehensive overview of the production of rigid polyurethane foams and their use as outer or core layer in composite elements and also their application as insulating layer in cooling or heating technology appears for example in “Polyurethane”, Kunststoff-Handbuch, volume 7, 3^{rd} edition, 1993, edited by Dr. Günter Oertel, Carl-Hanser-Verlag, Munich/Vienna.

Suitable rigid polyurethane foams are obtainable in known manner by reacting organic polyisocyanates with one or more compounds having two or more reactive hydrogen atoms in the presence of blowing agents, catalysts and optionally auxiliaries and/or additives.

The compounds used in the production of polyurethanes for their two or more isocyanate-reactive hydrogen atoms are preferably polyether alcohols and/or polyester alcohols. Polyols are selected with particular regard to costs and the desired performance characteristics (e.g., EP-A 1 632 511, U.S. Pat. No. 6,495,722, WO 2006/108833).

Isocyanate-based rigid foams are typically produced using polyols having high functionalities and a low molecular weight to optimally crosslink the foams. The preferably used polyether alcohols usually have a functionality of 4 to 8 and a hydroxyl number ranging from 300 to 600 and especially from 400 to 500 mg KOH/g. It is known that polyols having a very high functionality and hydroxyl numbers ranging from 300 to 600 have a very high viscosity. It is also known that polyols of this type are very polar and thus have poor dissolving power in respect of hydrocarbons. To remedy this defect, polyether alcohols having functionalities of 2 to 4 and hydroxyl numbers of 100 to 350 mg KOH/g are frequently added to the polyol component.

It is also known that the flowability of polyol components based on high-functionality, polar polyols is not always satisfactory. EP 1 138 709, however, discloses that rigid foams are preparable with good flowability when the polyol component comprises at least one polyether alcohol having a hydroxyl number of 100 to 250 mg KOH/g and obtained by addition of alkylene oxides onto H-functional starting substances having 2 to 4 active hydrogen atoms, especially glycols, trimethylolpropane, glycerol, pentaerythritol or TDA (tolylenediamine).

DE 198 12 174 describes a process for preparing polyester polyols using OH-containing fatty acid glycerides and their use for producing open-cell rigid polyurethane foams.

EP 1 923 417 discloses that a polyol component comprising polyetherester polyols based on fats having no OH groups, such as soya oil, have improved blowing agent solubilities and that the rigid foams produced therefrom have a short demolding time.

Foams obtainable by following the prior art described above fail to comply with all requirements.

It is an object of the present invention to provide a polyol component for producing rigid polyurethane foams which has a high solubility for physical blowing agents and is phase stable even under changes in composition. Phase stability shall be obtained by using the polyetherester polyols of the present invention. The use of polyetherester polyols having a higher blowing-agent solubility makes it possible to use formulations having a higher proportion of high-functionality crosslinker polyols, which should have higher compressive strength.

Such formulations shall further have a low viscosity and good processing properties, more particularly shall possess good flowability and enable rapid demolding.

We have found that this object is achieved by polyetherester polyols comprising the reaction product of
a1) 5 to 63 wt % of one or more polyols or polyamines or mixtures thereof having an average functionality of 2.5 to 8,
a2) 2 to 50 wt % of one or more fatty acids, fatty acid monoesters or mixtures thereof,
a3) 35 to 70 wt % of one or more alkylene oxides of 2 to 4 carbon atoms.

Using the polyetherester polyols of the present invention increases the network density of a resulting foam and thus improves its compressive strength. Structural components having a lower density but otherwise unchanged mechanical properties can be produced as a result.

The polyetherester polyols provide formulations for foams having increased compressive strengths and improved demolding properties. Surprisingly, these formulations display very good flow properties, even though low-viscosity polyols having a functionality of 2 to 4 and an OH number of less than 300 (i.e., so-called flowability polyols) are used in a very small amount, if at all.

The average functionality of the polyols, polyamines or mixtures of polyols and/or polyamines a1) is preferably in the range from 3 to 7 and more preferably in the range from 3.5 to 6.5.

Preferred polyols or polyamines of component a1) are selected from the group consisting of sugars and sugar alcohols (glucose, mannitol, sucrose, pentaerythritol, sorbitol), polyhydric phenols, resols, e.g. oligomeric condensation products of phenol and formaldehyde, trimethylolpropane, glycerol, tolylenediamine, ethylenediamine, ethylene glycols, propylene glycol and water. Particular preference is given to sugars and sugar alcohols such as sucrose and sorbitol, glycerol, water and ethylene glycols and also mixtures thereof, especial preference being given to mixtures comprising two or more compounds selected from sucrose, glycerol, water and diethylene glycol.

In one specific embodiment, component a1) comprises a mixture of glycerol and sucrose.

The proportion of the polyetherester polyols of the present invention which is contributed by polyols and/or polyamines a1) is generally in the range from 5 to 63 wt %, preferably in the range from 20 to 50 wt %, more preferably in the range from 30 to 40 wt %, and especially in the range from 32 to 38 wt %, based on the weight of polyetherester polyols.

In general, the fatty acid or fatty acid monoester a2) is selected from the group consisting of polyhydroxy fatty acids, ricinoleic acid, hydroxyl-modified oils, hydroxyl-modified fatty acids and fatty acid esters based on myristoleic acid, palmitoleic acid, oleic acid, stearic acid, palmitic acid, vaccenic acid, petroselic acid, gadoleic acid, erucic acid, nervonic acid, linoleic acid, α- and γ-linolenic acid, stearidonic acid, arachidonic acid, timnodonic acid, clupanodonic acid and cervonic acid. Methyl esters are preferred fatty acid monoesters. Preference is given to oleic acid, stearic acid, palmitic acid, linolenic acid and their monoesters and also mixtures thereof.

In one preferred embodiment of the invention, the fatty acids or fatty acid monoesters are used, used especially in the form of fatty acid methyl esters, biodiesel or pure fatty acids. Particular preference is given to biodiesel and pure fatty acids and specific preference to pure fatty acids, preferably oleic acid and stearic acid, especially oleic acid.

In a further preferred embodiment of the present invention, the fatty acid or fatty acid monoester a2) is oleic acid or stearic acid or a derivative of these fatty acids, particular preference being given to oleic acid, methyl oleate, stearic acid and methyl stearate. The fatty acid or fatty acid monoester is generally used to improve blowing agent solubility in the production of polyurethane foams.

The fatty acid and fatty acid monoester proportion of polyetherester polyols according to the present invention is generally in the range from 2 to 50 wt %, preferably in the range from 5 to 35 wt %, more preferably in the range from 8 to 30 wt % and especially in the range from 12 to 30 wt %, based on the weight of polyetherester polyols.

Useful alkylene oxides a3) have 2 to 4 carbon atoms and include for example tetrahydrofuran, 1,3-propylene oxide, 1,2-butylene oxide, 2,3-butylene oxide, styrene oxide and preferably ethylene oxide and 1,2-propylene oxide. The alkylene oxides can be used individually, alternatingly in succession or as mixtures. Propylene oxide and ethylene oxide are preferred alkylene oxides, mixtures of ethylene oxide and propylene oxide with >50 wt % of propylene oxide are particularly preferred, and pure propylene oxide is especially preferred.

One preferred embodiment utilizes an alkoxylation catalyst comprising an amine, preferably dimethylethanolamine or imidazole and more preferably imidazole.

The proportion of the polyetherester polyols of the present invention which is contributed by alkylene oxides is generally in the range from 35 to 70 wt %, preferably in the range from 38 to 65 wt %, more preferably in the range from 39 to 50 wt % and especially in the range from 40 to 45 wt %, based on the weight of the polyetherester polyols.

The OH number of the polyetherester polyols of the present invention is in the range from 300 to 800 mg KOH/g, preferably in the range from 400 to 700 mg KOH/g, more preferably in the range from 450 to 550 mg KOH/g and especially in the range from 475 to 550 mg KOH/g.

The average functionality of the polyetherester polyols of the present invention is generally in the range from 2.5 to 8, preferably in the range from 3 to 7, more preferably in the range from 3.5 to 6 and especially in the range from 4.5 to 5.5.

The viscosity of the polyetherester polyols of the present invention is generally <40 000 mPas, preferably <30 000 mPas, more preferably <2500 mPas and specifically <20 000 mPas, all measured at 25° C. to DIN 53018. Especially the use of methyl oleate as component a2) leads to a low viscosity.

The invention further provides a process for producing rigid polyurethane foams by reaction of

A) organic or modified organic polyisocyanates or mixtures thereof,

B) one or more of the polyetherester polyols of the present invention,

C) optionally polyester polyols,

D) optionally polyetherol polyols,

E) one or more blowing agents,

F) catalysts, and

G) optionally further auxiliaries and/or additives.

The present invention also provides a polyol mixture comprising said components B) to G), i.e.

B) one or more polyetherester polyols,

C) optionally polyester polyols,

D) optionally polyether polyols,

E) one or more blowing agents,

F) catalysts, and

G) optionally further auxiliaries and/or additives.

Further subjects of the present invention include rigid polyurethane foams, including rigid polyisocyanurate foams, obtainable via the process of the present invention and also the use of the polyetherester polyols of the present invention for producing rigid polyurethane foams.

The proportion of polyetherester polyols B) of the present invention is generally >25 wt %, preferably >40 wt %, more preferably >50 wt % and especially preferably >52 wt %, based on total components B) to G).

Production of rigid polyurethane foams by the process of the present invention, in addition to the specific polyetherester polyols described above, utilizes the constructal components known per se, which will now be detailed. Rigid polyurethane foams include rigid polyisocyanurate foams.

Possible organic polyisocyanates A) are the aliphatic, cycloaliphatic, araliphatic and preferably aromatic polyfunctional isocyanates known per se. The organic polyisocyanates may optionally be in a modified state.

Specific examples are: alkylene diisocyanates having from 4 to 12 carbon atoms in the alkylene radical, e.g. dodecane 1,12-diisocyanate, 2-ethyltetramethylene 1,4-diisocyanate, 2-methylpentamethylene 1,5-diisocyanate, tetramethylene 1,4-diisocyanate, and preferably hexamethylene 1,6-diisocyanate; cycloaliphatic diisocyanates such as cyclohexane 1,3- and 1,4-diisocyanate and also any mixtures of these isomers, 1-isocyanato-3,3,5-trimethyl-5-isocyanatomethylcyclohexane (IPDI), hexahydrotolylene 2,4- and 2,6-diisocyanate and also the corresponding isomer mixtures, dicyclohexylmethane 4,4′-, 2,2′- and 2,4′-diisocyanate and also the corresponding isomer mixtures, and preferably aromatic diisocyanates and polyisocyanates such as tolylene 2,4- and 2,6-diisocyanate and the corresponding isomer mixtures, diphenylmethane 4,4′-, 2,4′- and 2,2′-diisocyanate and the corresponding isomer mixtures, mixtures of diphenylmethane 4,4′- and 2,2′-diisocyanates, polyphenylpolymethylene polyisocyanates, mixtures of diphenylmethane 4,4′-, 2,4′- and 2,2′-diisocyanates and polyphenylpolymethylene polyisocyanates (crude MDI) and mixtures of crude MDI and tolylene diisocyanates. The organic diisocyanates and polyisocyanates can be used individually or in the form of their mixtures.

Preferred polyisocyanates are tolylene diisocyanate (TDI), diphenylmethane diisocyanate (MDI) and in particular mixtures of diphenylmethane diisocyanate and polyphenylenepolymethylene polyisocyanates (polymeric MDI or PMDI).

Use is frequently also made of modified polyfunctional isocyanates, i.e. products which are obtained by chemical reaction of organic polyisocyanates. Examples which may be mentioned are polyisocyanates comprising ester, urea, biuret, allophanate, carbodiimide, isocyanurate, uretdione, carbamate and/or urethane groups.

Very particular preference is given to using polymeric MDI for producing the rigid polyurethane foams of the invention.

Suitable polyester polyols C) can be prepared, for example, from organic dicarboxylic acids having from 2 to 12 carbon atoms, preferably aromatic or mixtures of aromatic and aliphatic dicarboxylic acids, and polyhydric alcohols, preferably dials, having from 2 to 12 carbon atoms, preferably from 2 to 6 carbon atoms. Possible dicarboxylic acids are, for example: succinic acid, glutaric acid, adipic acid, suberic acid, azelaic acid, sebacic acid, decanedicarboxylic acid, maleic acid, fumaric acid, phthalic acid, isophthalic acid and terephthalic acid. The dicarboxylic acids can be used either individually or in admixture with one another. It is also possible to use the corresponding dicarboxylic acid derivatives, e.g. dicarboxylic esters of alcohols having from 1 to 4 carbon atoms or dicarboxylic anhydrides, in place of the free dicarboxylic acids. As aromatic dicarboxylic acids, preference is given to using phthalic acid, phthalic anhydride, terephthalic acid and/or isophthalic acid as a mixture or alone. As aliphatic dicarboxylic acids, preference is given to using dicarboxylic acid mixtures of succinic, glutaric and adipic acid in weight ratios of, for example, 20-35:35-50:20-32, and in particular adipic acid. Examples of dihydric and polyhydric alcohols, in particular dials, are: ethanediol, diethylene glycol, 1,2- or 1,3-propanediol, dipropylene glycol, 1,4-butanediol, 1,5-pentanediol, 1,6-hexanediol, 1,10-decanediol, glycerol, trimethylolpropane and pentaerythritol. Preference is given to using ethanediol, diethylene glycol, 1,4-butanediol, 1,5-pentanediol, 1,6-hexanediol or mixtures of at least two of the dials mentioned, in particular mixtures of 1,4-butanediol, 1,5-pentane-dial and 1,6-hexanediol. It is also possible to use polyester polyols derived from lactones, e.g. E-caprolactone, or hydroxycarboxylic acids, e.g. co-hydroxycaproic acid.

To prepare the polyester polyols C), bio-based starting materials and/or derivatives thereof are also suitable, for example castor oil, palm oil, polyhydroxy fatty acids, ricinoleic acid, hydroxyl-modified oils, grapeseed oil, black cumin oil, pumpkin kernel oil, borage seed oil, soybean oil, wheat germ oil, rapeseed oil, sunflower oil, peanut oil, apricot kernel oil, pistachio oil, almond oil, olive oil, macadamia nut oil, avocado oil, sea buckthorn oil, sesame oil, hemp oil, hazelnut oil, primula oil, wild rose oil, safflower oil, walnut oil, hydroxyl-modified fatty acids and fatty acid esters based on myristoleic acid, palmitoleic acid, stearic acid, palmitic acid, oleic acid, vaccenic acid, petroselic acid, gadoleic acid, erucic acid, nervonic acid, linoleic acid, α- and γ-linolenic acid, stearidonic acid, arachidonic acid, timnodonic acid, clupanodonic acid and cervonic acid.

The level of polyester polyols C) is generally in the range from 0 to 25 wt %, based on total components B) to G). One preferred embodiment of the invention utilizes no further polyester polyols C).

Preferred polyester polyols C) are formed from adipic acid, phthalic anhydride and/or terephthalic anhydride as dicarboxylic acids and propylene glycol, dipropylene glycol, ethylene glycol, diethylene glycol, glycerol and/or trimethylolpropane as alcohol component as well as oleic acid or castor oil, and have an OH number in the range from 150 to 400 and a functionality in the range from 2 to 4.5.

It is also possible to make concomitant use of polyether polyols D) which are prepared by known methods, for example from one or more alkylene oxides having from 2 to 4 carbon atoms in the alkylene radical by anionic polymerization using alkali metal hydroxides, e.g. sodium or potassium hydroxide, or alkali metal alkoxides, e.g. sodium methoxide, sodium or potassium methoxide or potassium isopropoxide, as catalysts with addition of at least one starter molecule comprising from 2 to 8, preferably from 2 to 6, reactive hydrogen atoms, or by cationic polymerization using Lewis acids, e.g. antimony pentachloride, boron fluoride etherate, or bleaching earth, as catalysts.

Suitable alkylene oxides are, for example, tetrahydrofuran, 1,3-propylene oxide, 1,2- or 2,3-butylene oxide, styrene oxide and preferably ethylene oxide and 1,2-propylene oxide. The alkylene oxides can be used individually, alternately in succession or as mixtures. Preferred alkylene oxides are propylene oxide and ethylene oxide, with particular preference being given to propylene oxide.

Possible starter molecules are, for example: water, organic dicarboxylic acids, such as succinic acid, adipic acid, phthalic acid and terephthalic acid, aliphatic and aromatic, optionally N-monoalkyl-,N,N-dialkyl- and N,N′-dialkyl-substituted diamines having from 1 to 4 carbon atoms in the alkyl radical, e.g. optionally monoalkyl- and dialkyl-substituted ethylenediamine, diethylenetriamine, triethylenetetramine, 1,3-propylenediamine, 1,3- or 1,4-butylenediamine, 1,2-, 1,3-, 1,4-, 1,5- and 1,6-hexa-methylenediamine, phenylenediamines, 2,3-, 2,4- and 2,6-tolylenediamine and 4,4′-, 2,4′- and 2,2′-diaminodiphenylmethane.

Further possible starter molecules are: alkanolamines such as ethanolamine, N-methylethanolamine and N-ethylethanolamine, dialkanolamines, such as diethanolamine, N-methyldiethanolamine and N-ethyldiethanolamine, and trialkanolamines, such as triethanolamine, and ammonia.

Preference is given to using dihydric or polyhydric alcohols such as ethanediol, 1,2- and 1,3-propanediol, diethylene glycol, dipropylene glycol, 1,4-butanediol, 1,6-hexanediol, glycerol, trimethyloipropane, pentaerythritol, sorbitol and sucrose. Particular preference is given to the recited primary amines, for example 2,3-tolylenediamine.

The polyether polyols D), preferably polyoxypropylene polyols and/or polyoxyethylene polyols, have a functionality of preferably from 2 to 6 and in particular from 2 to 5 and number average molecular weights of from 150 to 3000, preferably from 200 to 1500 and in particular from 250 to 750.

A particularly preferred embodiment of the invention utilizes a propoxylated tolylenediamine, in particular 2,3-tolylenediamine, as polyether polyol D).

Useful polyether polyols further include polymer-modified polyether polyols, preferably grafted polyether polyols, especially grafted polyether polyols on a styrene and/or acrylonitrile base, which are formed by in situ polymerization of acrylonitrile, styrene or preferably mixtures of styrene and acrylonitrile, for example in a weight ratio of 90:10 to 10:90 and preferably 70:30 to 30:70, advantageously in the aforementioned polyether polyols as described in the German patent documents DE 11 11 394, 12 22 669 (U.S. Pat. Nos. 3,304,273; 3,383,351; 3,523,093), 11 52 536 (GB 1040452) and 11 52 537 (GB 987,618), and also polyether polyol dispersions where the disperse phase typically accounts for from 1 to 50 wt % and preferably 2 to 25 wt % and comprises for example polyureas, polyhydrazides, tert-amino-containing polyurethanes and/or melamine and which are described for example in EP-B 011 752 (U.S. Pat. No. 4,304,708), U.S. Pat. No. 4,374,209 and DE-A 32 31 497.

The polyether polyols can also be used in the form of mixtures. They can further be mixed with the polyester polyols or grafted polyether polyols as well as hydroxyl-containing polyesteramides, polyacetals, polycarbonates and/or polyether polyamines.

Useful hydroxyl-containing polyacetals include for example the compounds which can be prepared from glycols, such as diethylene glycol, triethylene glycol, 4,4′-dihydroxyethoxydiphenyldimethylmethane, hexanediol and formaldehyde. Suitable polyacetals are also obtainable by polymerizing cyclic acetals.

Useful hydroxyl-containing polycarbonates include those of the type known per se, which are obtainable for example by reacting diols, such as 1,3-propanediol, 1,4-butanediol and/or 1,6-hexanediol, diethylene glycol, triethylene glycol or tetraethylene glycol with diaryl carbonates, for example diphenyl carbonate, alkylene carbonate or phosgene.

Polyesteramides include for example the predominantly linear condensates obtained from polybasic, saturated and/or unsaturated carboxylic acids/anhydrides and polyfunctional saturated and/or unsaturated aminoalcohols or mixtures of polyfunctional alcohols and aminoalcohols and/or polyamines.

Suitable polyether polyamines are obtainable from the abovementioned polyether polyols by known methods. Examples are the cyanoalkylation of polyoxyalkylene polyols and subsequent hydrogenation of the nitrile obtained (U.S. Pat. No. 3,267,050), or the partial or complete amination of polyoxyalkylene polyols with amines or ammonia in the presence of hydrogen and catalysts (DE 12 15 373).

The proportion of polyether polyols D) is generally in the range from 75 to 55 wt %, preferably in the range from 55 to 30 wt % and more preferably in the range from 30 to 5 wt %, based on total components B) to G).

Blowing agents E) which are used for producing the rigid polyurethane foams include preferably water and physical blowing agents such as low-boiling hydrocarbons and mixtures thereof. Suitable physical blowing agents are liquids which are inert towards the organic, optionally modified polyisocyanates and have boiling points below 100° C., preferably below 50° C., at atmospheric pressure, so that they vaporize under the conditions of the exothermic polyaddition reaction. Examples of such liquids which can preferably be used are alkanes such as heptane, hexane, n-pentane and isopentane, preferably industrial mixtures of n-pentane and isopentane, n-butane and isobutane and propane, cycloalkanes such as cyclopentane and/or cyclohexane, ethers such as furan, dimethyl ether and diethyl ether, ketones such as acetone and methyl ethyl ketone, alkyl carboxylates such as methyl formate, dimethyl oxalate and ethyl acetate. Mixtures of these low-boiling liquids with one another and/or with other substituted or unsubstituted hydrocarbons can also be used. Organic carboxylic acids such as formic acid, acetic acid, oxalic acid, ricinoleic acid and carboxyl-containing compounds are also suitable.

It is preferable not to use formic acid or any halogenated hydrocarbons as blowing agent. It is preferable to use water, any pentane isomer and also mixtures of water and pentane isomers.

The blowing agents are wholly dissolved in the polyol component (i.e. B+C+E+F+G) or are introduced via a static mixer immediately before foaming of the polyol component.

The amount of physical blowing agent or blowing agent mixture used is in the range from 1 to 45 wt %, preferably in the range from 10 to 30 wt % and more preferably in the range from 10 to 20 wt %, all based on total components B) to G).

Water is preferably added, as blowing agent, to the component B) in an amount of 0.2 to 5 wt %, based on component B). The addition of water can take place in combination with the use of other blowing agents described. Preference is given to using water combined with pentane.

Catalysts F) used for preparing the rigid polyurethane foams are particularly compounds which substantially speed the reaction of the component B) to G) compounds comprising reactive hydrogen atoms, especially hydroxyl groups, with the organic, optionally modified polyisocyanates A).

It is advantageous to use basic polyurethane catalysts, for example tertiary amines such as triethylamine, tributylamine, dimethylbenzylamine, dicyclohexylmethylamine, dimethylcyclohexylamine, N,N,N′,N′-tetramethyldiaminodiethyl ether, bis(dimethyl-aminopropyl)urea, N-methylmorpholine or N-ethylmorpholine, N-cyclohexylmorpholine, N,N,N′,N′-tetramethylethylenediamine, N,N,N,N-tetramethylbutanediamine, N,N,N,N-tetramethylhexane-1,6-diamine, pentamethyldiethylenetriamine, dimethylpiperazine, N-dimethylaminoethylpiperidine, 1,2-dimethylimidazole, 1-azabicyclo-[2.2.0]octane, 1,4-diazabicyclo[2.2.2]octane (Dabco) and alkanolamine compounds, such as triethanolamine, triisopropanolamine, N-methyldiethanolamine and N-ethyldiethanolamine, dimethylaminoethanol, 2-(N,N-dimethylaminoethoxy)ethanol, N,N′,N″-tris(dialkylaminoalkyl)hexahydrotriazines, e.g. N,N′,N″-tris(dimethylaminopropyl)-s-hexahydrotriazine, and triethylenediamine. However, metal salts such as iron(II) chloride, zinc chloride, lead octoate and preferably tin salts such as tin dioctoate, tin diethylhexoate and dibutyltin dilaurate and also, in particular, mixtures of tertiary amines and organic tin salts are also suitable.

Further possible catalysts are: amidines such as 2,3-dimethyl-3,4,5,6-tetra-hydropyrimidine, tetraalkylammonium hydroxides such as tetramethylammonium hydroxide, alkali metal hydroxides such as sodium hydroxide and alkali metal alkoxides such as sodium methoxide and potassium isopropoxide, and also alkali metal salts of long-chain fatty acids having from 10 to 20 carbon atoms and optionally lateral OH groups. Preference is given to using from 0.001 to 5% by weight, in particular from 0.05 to 2% by weight, of catalyst or catalyst combination, based on the weight of the components B) to G). It is also possible to allow the reactions to proceed without catalysis. In this case, the catalytic activity of amine-initiated polyols is exploited.

When, during foaming, a relatively large polyisocyanate excess is used, further suitable catalysts for the trimerization reaction of the excess NCO groups with one another are: catalysts which form isocyanurate groups, for example ammonium ion salts or alkali metal salts, either alone or in combination with tertiary amines. Isocyanurate formation leads to flame-resistant PIR foams which are preferably used in industrial rigid foam, for example in building and construction as insulation boards or sandwich elements.

Further information regarding the abovementioned and further starting materials may be found in the technical literature, for example Kunststoffhandbuch, Volume VII, Polyurethane, Carl Hanser Verlag Munich, Vienna, 1st, 2nd and 3rd Editions 1966, 1983 and 1993.

Further auxiliaries and/or additives G) can optionally be added to the reaction mixture for producing the rigid polyurethane foams. Mention may be made of, for example, surface-active substances, foam stabilizers, cell regulators, fillers, dyes, pigments, flame retardants, hydrolysis inhibitors, fungistatic and bacteriostatic substances.

Possible surface-active substances are, for example, compounds which serve to aid homogenization of the starting materials and may also be suitable for regulating the cell structure of the polymers. Mention may be made of, for example, emulsifiers such as the sodium salts of castor oil sulfates or of fatty acids and also salts of fatty acids with amines, e.g. diethylamine oleate, diethanolamine stearate, diethanolamine ricinoleate, salts of sulfonic acids, e.g. alkali metal or ammonium salts of dodecylbenzenesulfonic or dinaphthylmethanedisulfonic acid and ricinoleic acid; foam stabilizers such as siloxane-oxyalkylene copolymers and other organopolysiloxanes, ethoxylated alkylphenols, ethoxylated fatty alcohols, paraffin oils, castor oil esters or ricinoleic esters, Turkey red oil and peanut oil, and cell regulators such as paraffins, fatty alcohols and dimethylpolysiloxanes. The above-described oligomeric acrylates having polyoxyalkylene and fluoroalkane radicals as side groups are also suitable for improving the emulsifying action, the cell structure and/or for stabilizing the foam. The surface-active substances are usually employed in amounts of from 0.01 to 5 wt %, based on the weight of components B) to G).

Fillers, in particular reinforcing fillers, are to be understood as meaning the customary organic and inorganic fillers, reinforcing materials, weighting agents, agents for improving the abrasion behavior in paints, coating compositions, etc., which are known per se. Specific examples are: inorganic fillers such as siliceous minerals, for example sheet silicates such as antigorite, serpentine, hornblendes, amphiboles, chrisotile and talc, metal oxides such as kaolin, aluminum oxides, titanium oxides and iron oxides, metal salts, such as chalk, barite and inorganic pigments such as cadmium sulfide and zinc sulfide and also glass, etc. Preference is given to using kaolin (china clay), aluminum silicate and coprecipitates of barium sulfate and aluminum silicate and also natural and synthetic fibrous minerals such as wollastonite, metal fibers and in particular glass fibers of various length, which may optionally have a coating of size. Possible organic fillers are, for example: carbon, melamine, rosin, cyclopentadienyl resins and graft polymers and also cellulose fibers, polyamide, polyacrylonitrile, polyurethane, polyester fibers based on aromatic and/or aliphatic dicarboxylic esters and in particular carbon fibers.

The inorganic and organic fillers can be used individually or as mixtures and are added to the reaction mixture in amounts of from 0.5 to 50 wt %, preferably from 1 to 40 wt %, based on the weight of the components A) to E), although the content of mats, nonwovens and woven fabrics of natural and synthetic fibers can reach values of up to 80 wt %.

Further information regarding the abovementioned other customary auxiliaries and additives may be found in the technical literature, for example the monograph by J. H. Saunders and K. C. Frisch “High Polymers” Volume XVI, Polyurethanes, Parts 1 and 2, Interscience Publishers 1962 and 1964, or Kunststoff-Handbuch, Polyurethane, Volume VII, Hanser-Verlag, Munich, Vienna, 1st and 2nd Editions, 1966 and 1983.

To produce the rigid polyurethane foams of the present invention, the optionally modified organic polyisocyanates A), the specific polyetherester polyols B) of the present invention, optionally the polyester polyols C) and optionally the polyetherols and/or further compounds having two or more isocyanate-reactive groups D) are reacted in such amounts that the equivalence ratio of NCO groups of the polyisocyanates A) to the sum of the reactive hydrogen atoms of the components B), optionally C), optionally D) and also E) and F) is in the range from 1 to 3:1, preferably in the range from 1.1 to 2:1 and more particularly in the range from 1. to 1.5:1.

In one preferred embodiment, the polyol component comprises

40 to 100 wt % of polyetherester polyols B),

0 wt % of further polyester polyols C),

5 to 40 wt % of polyether polyols D),

10 to 25 wt % of blowing agents E),

1.0 to 3 wt % of catalysts F), and

1 to 4 wt % of auxiliaries and/or additives G),

wherein the components B) and D) to G) sum to 100 wt %.

It is more preferable for the polyol component to comprise

50 to 80 wt % of polyetherester polyols B),

0 wt % of further polyester polyols C),

5 to 30 wt % of polyether polyols D),

10 to 20 wt % of blowing agents E),

1.0 to 2.5 wt % of catalysts F), and

1.5 to 3 wt % of further auxiliaries and/or additives G),

wherein the components B) and D) to G) sum to 100 wt %.

The rigid polyurethane foams are advantageously produced by the one shot process, for example using the high pressure or low pressure technique in open or closed molds, for example metallic molds. It is also customary to apply the reaction mixture in a continuous manner to suitable belt lines to produce panels.

The starting components are, at a temperature from 15 to 90° C., preferably from 20 to 60° C. and especially from 20 to 35° C., mixed and introduced into an open mold or, if necessary under superatmospheric pressure, into a closed mold. Mixing, as already noted, can be carried out mechanically using a stirrer or a stirring screw. Mold temperature is advantageously in the range from 20 to 110° C., preferably in the range from 30 to 70° C. and especially in the range from 40 to 60° C.

The rigid polyurethane foams produced by the process of the present invention have a density of 10 to 300 g/l, preferably of 15 to 100 g/l and especially of 20 to 40 g/l.

The invention is more particularly elucidated by the examples which follow.

### EXAMPLES

Pentane Solubility

Pentane solubility was determined by incrementally adding pentane to the component to be measured for pentane solubility. Pentane was added to exactly 100 g of the in-test component according to the likely pentane solubility, and mixed therewith. If the mixture was neither cloudy nor biphasic, further pentane had to be added and mixed in again.

When the mixture was biphasic, the glass was left to stand open to the atmosphere at room temperature until the excess pentane had evaporated and the remaining solution had become clear, and then the dissolved amount of pentane was weighed back.

In the event of cloudiness, the glass was sealed and left to stand at room temperature until two phases had formed. This was followed by evaporating and weighing back.

### Example 1

Pentane Compatibility of Inventive Polyetherester Polyols Versus Conventional Polyols

Sucrose/glycerol/PO polyol having an OH number of 450 and a functionality of 5.0 serves as comparative polyol. The inventive examples possess the stated proportion (in weight %) of fatty acid or fatty acid esters in addition to the starting materials sucrose/glycerol/PO.

TABLE 1

| | | | Pentane |
|---|---|---|---|
| | OH | | compatibility |
| Composition | number | Functionality | [%] |
| Glycerol(7.6%)-sucrose | 450 | 5.10 | 12.0 |
| (25.0%)-PO (67.4%); | | | |
| comparative example | | | |
| Glycerol(8.77%)-sucrose | 459.8 | 5.01 | 16.6 |
| (22.01%)-methyl oleate (5%)- | | | |
| PO (63.98%) | | | |
| Glycerol (8.77%)-sucrose | 450.8 | 5.02 | 25.9 |
| (22.07%)-methyl oleate (12%)- | | | |
| PO (57.11%) | | | |
| Glycerol(9.56%)-sucrose | 473.4 | 5.00 | 21.3 |
| (24.02%)-methyl oleate (25%)- | | | |
| PO (41.37%) | | | |
| Glycerol (6.48%)-sucrose | 455 | 5.02 | 16 |
| (24.5%)-oleic acid (5%)-PO | | | |
| (63.98%) | | | |
| Glycerol (4.89%)-sucrose | 447.4 | 5.02 | 21.9 |
| (26.19%)-oleic acid (8.5%)-PO | | | |
| (60.37%) | | | |
| Glycerol(3.33%)-sucrose | 459.6 | 5.01 | 30.0 |
| (27.84%)-oleic acid (12.01%)- | | | |
| PO (56.8%) | | | |

The comparative example shows that the pentane compatibility of polyols can be enhanced by using fatty acids and fatty acid monoesters.

### Comparative Example and Examples 2 and 3

The following components were reacted (all particulars in weight %):

polyol A from sugar 24.5%, glycerol 6.48%, PO 63.98%, oleic acid 5%, OH number 456 mg KOH/g;

polyol B from sugar 20.04%, glycerol 12.7%, PO 41.2%, methyl oleate 25.6%, OH number 489 mg KOH/g;

polyol C from vicinal TDA 24.9%, PO 75.1%, OH number 400 mg KOH/g;

polyol D from vicinal TDA 9.2%, EO 8.6%, PO 82.2, OH number 160 mg KOH/g;

polyol E is a polyetherol based on sucrose, glycerol and propylene oxide, KOH catalyzed, with a functionality of 5.1 and an OH number of 450 mg KOH/g;

stabilizer 1: silicone-containing foam stabilizer (Tegostab® B8474 from Evonik)

stabilizer 2: silicone-containing foam stabilizer (Tegostab® B8491 from Evonik)

catalyst 1: dimethylcyclohexylamine (DMCHA)

catalyst 2: pentamethyldiethylenetriamine (PMDETA)

catalyst 3: N,N,N-trisdimethylaminopropylhexahydrotriazine

catalyst 4: dimethylbenzylamine

isocyanate: polymer MDI with NCO content of 31.5 weight % (Lupranat® M20)

The stated raw materials (all particulars in weight %) were used to prepare a polyol component. Using a high-pressure Puromat® PU 30/80 IQ (Elastogran GmbH) with an output rate of 250 g/sec, the polyol component was mixed with the requisite amount of the stated isocyanate to obtain an isocyanate index (unless otherwise stated) of 116.7. The reaction mixture was injected into temperature-controlled molds measuring 2000 mm×200 mm×50 mm or 400 mm×700 mm×90 mm and allowed to foam up therein. Overpacking was 14.5%, i.e., 14.5% more reaction mixture was used than needed to completely foam out the mold.

TABLE 2

| | Comparative | | |
|---|---|---|---|
| | example | Example 2 | Example 3 |
| Polyol A | | 73 | |
| Polyol B | | | 60 |
| Polyol C | 18 | 11 | 30 |
| Polyol D | 15 | 6 | |
| Polyol E | 58 | | |
| Stabilizer 1 | 2 | 2 | 2 |
| Stabilizer 2 | 0.75 | 0.75 | 0.75 |
| H2O | 2.55 | 2.55 | 2.55 |
| Catalyst 1 | 0.57 | 0.618 | 0.44 |
| Catalyst 2 | 0.918 | 0.988 | 0.71 |
| Catalyst 3 | 0.459 | 0.494 | 0.35 |
| Cyclopentane 95% | 13 | 13 | 13 |
| NCO index | 116.7 | 118 | 118 |
| Fiber time [s] | 38 | 37 | 37 |
| Free rise density [g/L] | 24.99 | 22.73 | 23.4 |
| Polyol blend stability | clear | clear | clear |
| with cyclopentane at | | | |
| RT | | | |
| Polyol blend stability | clear | clear | clear |
| with cyclopentane at | | | |
| 6° C. | | | |
| Post-expansion | | | |
| [%] | | | |
| 3 min | 3.6 | 3.56 | 3.11 |
| 4 min | 2.17 | 2.33 | 1.78 |
| 5 min | 1.44 | 1.56 | 1.00 |
| 7 min | 0.55 | 0.56 | 0.33 |
| Thermal conductivity | 18.83 | 18.83 | 19.1 |
| [mW/m * K] | | | |
| Flowability | 1.31 | 1.31 | 1.28 |
| Compressive strength | 0.152 | 0.160 | 0.144 |
| [N/mm²]@ 31 g/L | | | |

Example 2 versus the formulation of Comparative Example 1 surprisingly shows 5.26% higher compressive strength coupled with unchanged flow and demolding properties. This was unforeseeable because a person skilled in the art knows that the increased use of crosslinker polyols and the reduced proportion of flowability polyol leads to inferior flow properties.

Example 3 versus Comparative Example 1 surprisingly shows a 0.22%-0.49% lower post-expansion and hence an improvement in demolding properties and also an improved flowability.

These described advantages result from the specific chemical structure of polyetherester polyols according to the present invention and the formulation freedom gained as a result and also from the possibility of preparing novel formulations compatible with blow agents.

### Example 4

20.2 g of glycerol, 0.1 g of imidazole, 50.8 g of sucrose as well as 11.5 g of methyl oleate were initially charged to a 300 ml reactor at 25° C. The reactor was then inertized with nitrogen. The kettle was heated to 130° C. and 147.5 g of propylene oxide were metered in. Following a reaction time of 9 h, the kettle was fully evacuated at 100° C. for 30 minutes and then cooled down to 25° C. to obtain 217 g of product.

The polyetherester obtained had the following characteristic values:

OH number: 459.8 mg KOH/g

Viscosity (25° C.): 11 324 mPas

Acid number: less than 0.01 mg KOH/g

Water content: less than 0.01%

### Example 5

### Producing a Polyetherester with Methyl Oleate

20.2 g of glycerol, 0.1 g of imidazole, 50.8 g of sucrose as well as 27.6 g of methyl oleate were initially charged to a 300 ml reactor at 25° C. The reactor was then inertized with nitrogen. The kettle was heated to 130° C. and 131.4 g of propylene oxide were metered in. Following a reaction time of 5 h, the kettle was fully evacuated at 100° C. for 40 minutes and then cooled down to 25° C. to obtain 219 g of product.

The polyetherester obtained had the following characteristic values:

OH number: 450.8 mg KOH/g

Viscosity (25° C.): 9453 mPas

Acid number: 0.05 mg KOH/g

Water content: 0.04%

### Example 6

### Producing a Polyetherester with Methyl Oleate

477.9 g of glycerol, 2.5 g of imidazole, 1250.2 g of sucrose as well as 1250.2 g of methyl oleate were initially charged to a 5 L reactor at 25° C. The reactor was then inertized with nitrogen. The kettle was heated to 130° C. and 2068.5 g of propylene oxide were metered in. Following a reaction time of 3.5 h, the kettle was fully evacuated at 100° C. for 60 minutes and then cooled down to 25° C. to obtain 4834.8 g of product.

The polyetherester obtained had the following characteristic values:

OH number: 473.4 mg KOH/g

Viscosity (25° C.): 11 892 mPas

Acid number: 0.17 mg KOH/g

Water content: 0.021%

### Example 7

### Producing a Polyetherester with Oleic Acid

14.9 g of glycerol, 0.1 g of imidazole, 56.4 g of sucrose as well as 11.6 g of oleic acid were initially charged to a 300 mL reactor at 25° C. The reactor was then inertized with nitrogen. The kettle was heated to 130° C. and 147.1 g of propylene oxide were metered in. Following a reaction time of 7 h, the kettle was fully evacuated at 100° C. for 40 minutes and then cooled down to 25° C. to obtain 216.9 g of product.

The polyetherester obtained had the following characteristic values:

OH number: 455 mg KOH/g

Viscosity (25° C.): 20 212 mPas

Acid number: less than 0.01 mg KOH/g

Water content: less than 0.01%

### Example 8

### Producing a Polyetherester with Oleic Acid

244.4 g of glycerol, 2.5 g of imidazole, 1309.5 g of sucrose as well as 425.1 g of oleic acid were initially charged to a 5 L reactor at 25° C. The reactor was then inertized with nitrogen. The kettle was heated to 130° C. and 3019.1 g of propylene oxide were metered in. Following a reaction time of 4.5 h, the kettle was fully evacuated at 100° C. for 40 minutes and then cooled down to 25° C. to obtain 4926.8 g of product.

The polyetherester obtained had the following characteristic values:

OH number: 447.4 mg KOH/g

Viscosity (25° C.): 20 477 mPas

Acid number: less than 0.01 mg KOH/g

Water content: less than 0.03%

### Example 9

### Producing a Polyetherester with Oleic Acid

7.7 g of glycerol, 0.1 g of imidazole, 64.0 g of sucrose as well as 27.6 g of oleic acid were initially charged to a 300 mL reactor at 25° C. The reactor was then inertized with nitrogen. The kettle was heated to 130° C. and 130.6 g of propylene oxide were metered in. Following a reaction time of 7 h, the kettle was fully evacuated at 100° C. for 30 minutes and then cooled down to 25° C. to obtain 211.9 g of product.

The polyetherester obtained had the following characteristic values:

OH number: 459.6 mg KOH/g

Viscosity (25° C.): 41 321 mPas

Acid number: less than 0.13 mg KOH/g

Water content: less than 0.01%

### Example 10

### Producing a Polyetherester with Methyl Oleate

50.7 kg of glycerol, 0.2 kg of imidazole, 81.8 kg of sucrose as well as 102.4 kg of methyl oleate were initially charged to a 600 L reactor at 25° C. The reactor was then inertized with nitrogen. The kettle was heated to 120° C. and 165.0 kg of propylene oxide were metered in. Following a reaction time of 4 h, the kettle was fully evacuated at 120° C. for 30 minutes and then cooled down to 25° C. to obtain 377.0 kg of product.

The polyetherester obtained had the following characteristic values:

OH number: 458.0 mg KOH/g

Viscosity (25° C.): 8783 mPas

Acid number: less than 0.01 mg KOH/g

Water content: less than 0.01%

### Example 11

### Producing a Polyetherester with Oleic Acid

25.9 kg of glycerol, 0.2 kg of imidazole, 98.0 kg of sucrose as well as 20.1 kg of oleic acid were initially charged to a 600 L reactor at 25° C. The reactor was then inertized with nitrogen. The kettle was heated to 120° C. and 255.8 kg of propylene oxide were metered in. Following a reaction time of 1 h, the kettle was fully evacuated at 120° C. for 30 minutes and then cooled down to 25° C. to obtain 390.0 kg of product.

The polyetherester obtained had the following characteristic values:

OH number: 456.0 mg KOH/g

Viscosity (25° C.): 17 367 mPas

Acid number: less than 0.01 mg KOH/g

Water content: less than 0.01%

We claim:

1. A polyetherester polyol comprising the reaction product of
a1) 5 to 63 wt % of one or more polyols or polyamines or mixtures thereof having an average functionality of 2.5 to 8,
a2) 2 to 50 wt % of one or more fatty acids, fatty acid monoesters or mixtures thereof,
a3) 35 to 70 wt % of one or more alkylene oxides of 2 to 4 carbon atoms.

2. The polyetherester polyol according to claim 1 wherein the polyols or polyamines of component a1) are selected from the group consisting of sugars, pentaerythritol, sorbitol, trimethylolpropane, glycerol, tolylenediamine, ethylenediamine, ethylene glycol, propylene glycol and water.

3. The polyetherester polyol according to claim 2 wherein said component a1) comprises a mixture of glycerol and sucrose.

4. The polyetherester polyol according to claim 2 wherein said component a2) comprises oleic acid, stearic acid, palmitic acid, linolenic acid, their monoesters or mixtures thereof.

5. The polyetherester polyol according to claim 1 wherein the alkylene oxide of component a3) is propylene oxide.

6. The polyetherester polyol according to claim 1 wherein it has an OH number of 200 to 700 mg KOH/g.

7. The polyetherester polyol according to claim 1 wherein it has a functionality of 2.5 to 8.

8. A process for producing rigid polyurethane foams by reaction of
A) organic or modified organic polyisocyanates or mixtures thereof,
B) one or more polyetherester polyols according to claim 1,
C) optionally further polyester polyols,
D) optionally polyetherol polyols,
E) one or more blowing agents,
F) catalysts, and
G) optionally further auxiliaries and/or additives.

9. A rigid polyurethane foam obtainable by the process according to claim 8.

10. A polyol mixture comprising as components
B) one or more polyetherester polyols according to claim 1,
C) optionally polyester polyols,
D) optionally polyetherol polyols,
E) one or more blowing agents,
F) catalysts, and
G) optionally further auxiliaries and/or additives.

11. The polyol mixture according to claim 10 comprising
50 to 80 wt % of polyetherester polyols B),
5 to 30 wt % of polyether polyols D),
10 to 20 wt % of blowing agents E),
1.0 to 2.5 wt % of catalysts F),
1.5 to 3 wt % of further auxiliaries and/or additives G),
wherein said components B) and D) to G) sum to 100 wt %.

12. The polyol mixture according to claim 10 comprising no further polyester polyols C).

13. The polyol mixture according to claim 10 comprising propoxylated tolylenediamine as polyether polyol D).
